(19) United States
(12) Patent Application Publication
(10) Pub. No.: US 2005/0065208 A1
(43) Pub. Date: Mar. 24, 2005

(54) Benzofuranes and their use in the treatment of atrial fibrillation

(76) Inventors: Bodo Brandts, Bochum (DE); Bo Carlson, Stockholm (SE); Johan Malm, Trangsund (SE)

Correspondence Address: WIGGIN AND DANA LLP;ATTENTION: PATENT DOCKETING, ONE CENTURY TOWER, P.O. BOX 1832, NEW HAVEN, CT, 06508-1832, US

(21) Appl. No.: 10/483,827

(22) PCT Filed: Jul. 15, 2002
(86) PCT No.: PCT/EP02/07905

(30) Foreign Application Priority Data
Jul. 20, 2001 (GB) 0117778.1
Jul. 20, 2001 (GB) 0117780.7

(51) Int. Cl. C07D 307/87; A61K 31/343

(52) U.S. Cl. 514/469; 549/462

(57) ABSTRACT

This intention relates to new compounds and their pharmaceutical use, and to the pharmaceutical use of known compounds, which compounds inhibit certain transmembrane potassium currents in the atrium of the heart of a mammal without significantly affecting other ion channels, for the treatment of heart disease particularly atrial fibrillation. The invention also relates to pharmaceutical compositions comprising such compounds.

### FIELD OF THE INVENTION

This invention relates to novel compounds that inhibit certain transmembrane potassium currents in the atrium of the heart of a mammal without significantly affecting other ion channels. It also relates to the use of certain known compounds in the, preparation of a medicament for the treatment of heart diseases, particularly atrial fibrillation. It further relates to pharmaceutical compositions containing compounds that inhibit certain transmembrane potassium currents in the atrium of the heart of a mammal without significantly affecting other ion channels, for the treatment of heart disease, particularly atrial fibrillation.

### BACKGROUND OF THE INVENTION

Cell membranes have a basic lipid bilayer structure that is impermeable to ions. Special proteins (hereafter referred to as ion-channels) have evolved that provide pathways for ions to cross cell membranes and so make the membrane permeable to ions, such as potassium (hereafter K), as sodium (hereafter Na) or calcium (hereafter Ca). Opening and closing of ion-channels make the membrane permeable or impermeable to different ions and thereby they regulate many properties and functions of the cell membrane. Ion-channels enable cells to set up membrane potentials, and allow currents to flow that change these membrane potentials, thereby underlying electrical signaling by the cell membrane. A transmembrane current (hereafter I) is the ion-flow through an open ion-channel. Ion-channels are targets for many antiarrhythmic drugs, which are used to treat abnormal electrical activity in the heart. From a therapeutic perspective, blocking of K-channels prolongs the action potential duration (APD) and lengthens the refractory period, and is a classical antiarrhythmic mechanism generating a Q-T prolongation on the surface ECG (Singh B and Nademanee K, Am Heart J, 1985, 109:421-30).

Several different kinds of ion-channels, including Na— Ca— and K— ion channels, are active in the mammalian heart giving rise to different ion-currents (e.g. INa, ICa and IK). Most K-channels are either voltage activated such as the Delayed Rectifier K-channel (resulting in the current IK), the Transient Outward K-channel (resulting in the current Ito) or ligand operated such as the ATP-sensitive K-channel which is opened during metabolic impairment (when intracellular levels of ATP are reduced) which generates the current IK(ATP). Another ligand-activated K-channel is the Muscarinic K-channel which is activated when acetylcholine binds to the muscarinic receptor M2 (resulting in the current IK(ACh) or when adenosine binds to the adenosine receptor A1 in the current IK(Ado).

Antiarrhythmic drugs are grouped according to their essential inhibitory effects on certain ion-currents; class I drugs predominantly inhibit sodium currents and class III drugs predominantly inhibit potassium currents. However, antiarrhythmic drugs that are used today are not selective in their ion-channel blocking and every drug used today interacts with several currents.

K-channel blocking in the heart may be one of the most efficient antiarrhythmic mechanisms identified so far. The problem is that any drug that prolongs repolarization has an intrinsically associated risk of inducing torsade de points arrhythmia in the ventricle. However, since the K-channels responsible for repolarization actually differ between the atrium and the ventricle, it is possible to identify K-channels that will be active against supraventricular arrhythmias but that will not prolong the QT-interval and thus will not be proarrhythinic.

Blocking of the particular ligand-activated K-currents IK(Ado) and/or IK(ACh) has been shown to occur with anti-arrhythmic agents. It has also been postulated that this mechanism may be of importance in explaining the efficacy of anti-arrhythmic drugs for the treatment of atrial fibrillation (Mori K, et al. Circulation 1995 Jun. 1;91(11):2834-43; Ohmoto-Sekine Y, et al. Br J Pharmacol 1999 February;126(3):751-61; Watanabe Y, et al. J Pharmacol Exp Ther 1996 November;279(2):617-24). The ligand-gated currents IK(Ado), IK(ACh) and Il((ATP) probably only have minor roles in shaping repolarization under normal conditions but, when activated by extracellular acetylcholine, by extracellular adenosine or reduction of intracellular ATP concentrations respectively, these currents are increased and thus can substantially shorten the action potential duration (APD) (Belardinelli L, et al. FASEB J 1995; 9(5):359-365; Belardinelli L and Isenberg G. Am J Physiol 1983; 244(5):H734-H737; Findlay I and Faivre J F. FEBS Lett 1991; 279(1):95-97). The therapeutic effect of anti arrhythmic agents is to prolong APD and thereby make the atrial myocardium more refractive to abnormal electrical activity.

It is expected that the ligand-gated channels IK(Ado) and IM(ATP) are-more active in atrial tachyarrhythmias (i.e. atrial fibrillation (AF) and atrial flutter) than in normal sinus-rhythm, whereas IK(ACh), activation is dependent on vagal activity (presynaptic release of ACh). Atrial consumption of ATP is increased in atrial tachyarrhythmias leading to increased levels of adenosine (a metabolite of ATP) activating IK(Ado) and leading to reduced intracellular ATP concentration, hence, activating IK(ATP) (Asheroft S J and Ashcroft F M. Cell Signal 1990; 2(3):197-214).

Atrial fibrillation is today seldom treated with antiarrhythmic agents to normalize the abnormal electric activity. The primary reason for the reluctance to treat AF-patients with drugs that effectively normalize atrial electric activity is that available anti-arrhythmic drugs also block other ion-channels, in addition to the ligand-gated channels IK(Ado), IK(ACh) and IK(ATP), in the heart. Therefore, treatment of AF-patients with currently-available anti-arrhythmic drugs is associated with a substantial risk to induce lethal proarrhythmic effects (as Torsade-de Points in the ventricle); It is of importance to consider that the antiarrhythmic agents referenced in Table 1 are not exclusively active on the ligand-gated currents IK(Ado), IK(ACh) and IK(ATP), but also block other transmembrane currents (references in Table 2).

The class III-agent amiodarone has been shown to be effective for treatment of AF (Roy D, et al., N Engl J Med 2000 Mar. 30;342(13):913-20) and indeed aniodarone does block ligand-gated currents IK(Ado) and IK(ACh) (Watanabe Y, et al. supra). However, in spite of the proven efficacy of amiodarone to treat AF, the side effect profile of the drug is complex; there are features such as pulmonary toxicity, ocular and skin changes, and other forms of organ toxicity that clearly limit its widespread clinical utility (Pollak, T. M. Am. J. Cardiol., 1999, 84, 37R-45R; Wiersinga, W. M. Chapter 10, Amiodarone and the Thyroid, In Handbook of Experimental Pharmacology, Weetman A. P., Grossman, A., Eds.; Springer-Verlag.: Berlin, Heidelberg, 1997, Vol 128). Amiodarone has a complex pharmacokinetic profile and the elimination of the drug is extremely slow (Wiersingha, supra). In spite of its proven efficacy for treatment of AF, amiodarone is not frequently used as a treatment due to all side effects associated with its use. A novel anti-arrhytmic drug which shares the inhibitory effect on the ligand activated currents IK(Ado)/IK(ACh) with amiodarone but displays lower organ toxicity than that drug would provide an improved treatment for AF. Indeed, data from toxicological studies performed with compounds of the, present invention or used in the present invention suggest a reduced toxicity as compared to amiodarone. The extreme pharmacokinetic behavior amiodarone complicates dosing of that drug and thus it would be of great clinical benefit to have a drug which shares the inhibitory effects on the; ligand activated currents IK(Ado)/IK(ACh)/IK(ATP) with amiodarone but that displays mainstream pharmacokinetics. Data from blood pharmacokinetics, tissue distribution and mass balance studies on compounds used in the present invention indicates that the clinical use of these compounds will be less complicated than that of amiodarone. An ideal drug for treatment of atrial fibrillation should also selectively inhibit the atrial currents that are increased under the pathological conditions characterizing the disease and lack effects on other currents. This is the case with the compounds of the present invention since the IK(Ado)/ATP current is predominantly active in the fibrillating atrium and the IK(ACh) is the current responsible for the induction of vagal-triggered atrial fibrillation. In comparison with other antiaarhythmic drugs (see table 2) the compounds of the present invention are essentially free from interactions with other ion-currents and can therefore be regarded as selective inhibitors of the K-currents (IK(Ado), IK(ACh) and IK(ATP)) that have an increased activity in supraventricular cardiac arrhytmias (i.e. atrial fibrillation) but without the ability to block the ion-currents that mediate electrical activity in the cardiac ventricles and in the normal atrium.

Both the compounds that are the subject of the present invention and amiodarone have been shown to antagonize triiodthyronine (T3)-signalling action in cells (manuscript in preparation) and therefore it should be noted that the inhibitory effects seen with such compounds on IK(Ado), IK(ACh) and IK(ATP)) are not due to T3-antagonism. There are two findings that support this statement; a) T3 does not have acute effects on IK(Ado) or IK(ACh) and b) potent T3-antagonists (100× more potent than the compounds that are the subject of the present invention on T3-receptor mediated signaling) do not display similar acute effects on IK(Ado) or IK(ACh).

### DESCRIPTION OF THE INVENTION

In the present invention acute and chronic effects of various compounds have been investigated by using electrophysiology technique applied to cardiomyocyte cultures. The inventions have found that certain compounds inhibit transmembrane K-currents that are induced through stimulation by muscarinic receptor agonists such as AcetylCholine (ACh) or A1 adenosine receptor agonists such as Adenosine (Ado) and by reduction of intracellular ATP.

The inhibitory effects occur within seconds after induction of the current with ACh, Ado or dinitrophenole (DNP reduces intracellular ATP). The acute inhibitory effects caused by the compounds of the present invention on these K-currents in cardiac muscle tissue had not previously been discovered. The reasons for this include the fact that these ligand activated K-currents (IK(Ado), IK(ACh) and IK(ATP)) are preferentially active in the atrial cardiomyocytes (Workman A J et al. Cardiovasc Res 1999 September;43(4):974-84; Koumi S-I, and Wasserstorm A. American Journal of Physiology 266[35], H1812-H1821. 1994), while previous studies have been carried out with tissue from cardiac ventricles. Furthermore, IK(Ado) and IK(ACh) must first be induced via the M2 or A1 receptor (with ACh and Ado respectively) before any inhibition can be observed. Without any agonist at the extracellular site of the membrane these ligand-gated channels probably have only minor roles in shaping repolarization but, when activated by extracellular acetylcholine or adenosine, they can substantially shorten action potential duration in the atrium (Tristani-Firouzi M et al. Am J Med 2001 January; 110(1):50-9).

Similar effects (i.e. inhibition of IK(Ado) or IK(ACh)) have been described for other antiarrhythmic drugs such as: E-4031, and MS-551 (Mori et al. supra), aprinidine (Ohmoto et al. supra) Amiodarone (Watanabe et al. supra) and terikalant (Brandts B. et al. Pacing Clin Electrophysiol 2000 November;23(11 Pt 2):1812-5); see Table 1.

One aspect of the invention is that compounds that are able to block one or both of the K-currents IK(Ado) and IK(ATP) should be efficient as pharmacological treatments for atrial fibrillation and/or atrial flutter.

It is wells known that prolonged atrial fibrillation facilitates he persistence and/or reoccurrence of arrhythmia (Wijffels M. et al. Circulation 92, 1954-1968. 1995). The pathophysiological background of this observation is the alteration of ion channel expression in atrial myocytes (electrical remodeling; Yue L. et al. Circulation Research 81, 512-525. 1997; Yue L. et al. Circ Res 1999; 84(7):(776-784). Seeking for strategies to treat atrial fibrillation one has to appreciate the fact that electrical remodeling is not the primary cause of the arrhythmia. Electrical remodeling is, a phenomenon that develops in patients and in the healthy heart. Other mechanisms than electrical remodeling are suggested to be responsible for the development of the “disease atrial fibrillation”. These mechanisms are discussed to be relevant at the early phase of the arrhythmia (a few minutes to a few hours).

The high frequency activation of the atrial myocardium during atrial fibrillation (more than 5 Hz) is suggested to significantly increase atrial oxygen consumption and thereby to significantly increase intracellular and interstitial adenosine concentrations due to intracellular loss of ATP. These mechanisms have been well described for ventricular fibrillation (Weiss J N et al. J Physiol 1992; 447:649-673; Schrader J. et al. Experientia 1990; 46(11-12):1172-1175; Decking U K et al. Circ Res 1997; 81(2):154-164; Deussen A. and Sclirader J. J Mol Cell Cardiol 1991; 23(4):495-504). Due to methodical difficulties at the atrial level (much less tissue, no option to selectively collect atrial effluate) only indirect observations suggest the occurrence of ischaemia during atrial fibrillation. After episodes of atrial fibrillation Daod et al. showed a reduction of atrial effective refractory period which was abolished after some tens of seconds during sinus rhythm (Daoud E G et al. Circ 1996; 94:1600-1606). Furthermore Rubart et al. showed elevated potassium concentrations during AF (Rubart M. et al. J Cardiovasc Electrophysiol 2000; 11 (6):652-664). Both observations fit very well with the hypothesis of atrial fibrillation-induced ischaemia in the atria. The consequence of atrial ischaemia during atrial fibrillation would be the activation of IK(Ado) and IK(ATP). Both currents are known to markedly reduce the atrial effective refractory period. A reduction of this period however is known to be one major determinant for the development of reentry tachycardias like atrial fibrillation. Since inhibition of IK(ATP) and IK(Ado) could reverse the shortening of the atrial effective refractory period such an inhibition is expected to be of significant pharmacological value in the treatment of atrial fibrillation. Moreover, since the ventricular tissue is activated at a “normal” rate during atrial fibrillation IK(Ado) and IK(ATP) are not expected to be active. Hence a drug which selectively inhibits IK(Ado) and IK(ATP) will not influence ventricular electrophysiolgy, and hence will not exert dangerous proarrhythmic effects. Furthermore, as mentioned above, IK(Ado) is much less expressed in ventricular myocytes.

Another aspect of the invention is the fact that compounds that are able to block. IK(ACh) should be efficient as pharmacologial treatments for a defined subgroup of patients in which the pathophysiology of atrial fibrillation has been well defined: Vagal-induced atrial fibrillation is regarded as an arrhythmia occurring when an increased vagal activity reduces the atrial effective refractory period by activation of IK(ACh). Because adenosine- and acetylcholine-induced inward rectifying potassium current is represented by the activation of the same ion channel population (Bünemann M. et al. J Physiol (Lond) 1995; 489(3):701-707; Bünemann M. et al. EMBO 1996), an inhibitor of adenosine-activated ion channels will also be an effective inhibitor of IK(ACh). Inhibition of IK(ACh) would be of significant value for the treatment of vagal-induced atrial fibrillation.

There is a unique specificity of the compounds that are the subject of the invention to exclusively block the three currents IK(ACh), IK(Ado), and IK(ATP). Several compounds that display well-known anti-arrhythmic properties have been shown to inhibit at least one of these three currents (see Table 2). However, all these other compounds are known to inhibit other ion-currents as well. Table 2 is a compilation of antiarrhythmic drugs that have been shown to inhibit IK(ACh). Interestingly compounds from different classes of antiarrhythmic compounds have all been shown to display similar actions on this particular current and the compilation includes “second generation” class III antiarrhythmic compounds, such as D-sotalol and Terikalant, which are potent inhibitors of the rapid component of delayed rectifying K-current (IKr). The compilation also includes the class III agents Amiodarone and Dronedarone that are known to inhibit several transmembrane currents (i.e Ca-currents) in addition to the currents listed in table 2. Also class I antiarrhythmic drugs as Flecainide, Quinidine, Disopyramide and Aprinidine are included. The most prominent mechanisms of antiarrhythmic activity of these class I compounds blockade of inward Na-currents.

Results from voltage clamp experiments with compounds of the invention on other ion-currents than IK(Ado), IK(ACh) and IK(ATP) are included in Table 2. Data from these voltage-clamp experiments demonstrate the absence of any relevant inhibition of the currents IK1, IKs, INa and Ito by compounds of the present invention.

The unique selectivity of the compounds that are the subject of the present invention to solely inhibit IK(ACh), IK(Ado), and IK(ATP) suggests that they will be effective in the treatment of atrial fibrillation and/or atrial flutter to normalize pathological electric activity in the atrium. The absence of inhibition of other ion-currents such as the inward rectifier (IK1), the slow component of the delayed rectifier (IKs), the transient outward K-current (Ito) or the depolarizing Na-current (INa) predict the risks for the compounds of the present invention to induce proarrhytmicity in normal cardiac tissue to be minor. Today clinicians are reluctant to treat AF-patients with effective antiarrhythmic drugs due to the intrinsic risks of proarrhythmic effects in the ventricles associated with the currently-available drugs. The selective action of the compounds of the present invention excludes significant effects on ventricular electrophysiology yielding prevention of proarrhythmias at that level. Moreover, the pharmacodynamic profile of the compounds of the present invention is expected to be of special value for the treatment of every kind of atrial fibrillation (inclusive of vagal-induced atrial fibrillation) without ventricular proarrhythmias.

Another aspect of the invention is that the compounds that it is concerned with are at least as potent as the drug amiodarone as blockers of the currents IK(Ado), IK(Ach) and IK(ATP) and this aspect together with the available safety documentation on the compounds of the present invention, suggesting an apparently much better safety profile than what is seen with amiodarone, indicates that the compounds of the present invention will be at least as efficaceous as amiodarone for treatment of AF but with fewer adverse effects.

### DETAILED DESCRIPTION OF THE INVENTION

In accordance with a first, of the present invention, novel compounds are provided that inhibit certain transmembrane K-currents that are induced through stimulation by muscarinic receptor agonists such as AcetylCholine (ACh) or A1 adenosine receptor agonists such as Adenosine (Ado) and by reduction of intracellular ATP.

Consequently, in a first aspect of the invention there are provided compounds according to the general formula I: 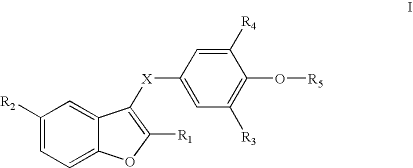
wherein;
R₁ is C₁-C₄ alkyl;
R₂ is NHCOR^{a}, NHCONHR^{a}, or hydrogen;
R₃ and R are independently selected from fluorine, chlorine, C₁-C₆ alkyl, and CF₃;
R^{a} is selected from CF₃, C₁₋₃ alkyl, and -(4-R^{b})C₆H₄;
R^{b} is selected from C₁₋₄ alkoxy, hydroxy, fluoro, and nitro;
R₅ is selected from hydrogen and —CH₂—COOH;
X is selected from CH₂ and C═O; with the proviso that when R₅ is hydrogen, X is —CH₂—;
   and pharmaceutically acceptable salts, esters and isomers thereof.

Preferably R₂ is hydrogen. Also preferably, where R₂ is H or NHCOR^{a}, R₃ and R₄ are independently C₁-C₄ alkyl, and more preferably R₃ and R₄ are both isopropyl.

In compounds where R₅ is —CH₂—COOH, R₁ is preferably methyl; R₂ is preferably hydrogen; R₃ and R₄ are preferably independently C₁-C₄ alkyl; R₅ is preferably —CH₂—COOH; and X is preferably —CH₂—.

Especially preferred compounds of the invention are:
2-methyl-3-(3,5-diisopropyl-4-hydroxybenzoyl)benzofuran (E1);
2-methyl-3-(3,5-diisopropyl-4-carboxymethoxybenzoyl)benzofuran (E2);
2-methyl-3-(3,5-diisopropyl-4hydroxybenzyl)benzofiuran (E3);
2-methyl-3-(3,5-diisopropyl-4carboxymethoxybenzyl)benzofuran (E4);
   and pharmaceutically acceptable salts and esters thereof and isomers thereof.

In accordance with a second aspect of the invention there is provided a pharmaceutical use of a compound that inhibits certain transmembrane potassium current, which are more active in the diseased atrium of a mammalian heart than in a normal atrium, without affecting other ion channels, for the preparation of a medicament for the treatment or prevention of atrial fibrillation and atrial flutter. Preferably the said inhibition derives from inhibition of one or several of the three ligand-sensitive potassium currents IK(Ado), IK(ACh) and IK(ATP). The inhibition caused by the said compound is more preferably not due to a T3 antagonistic effect.

The said compounds are described by the general formula II: 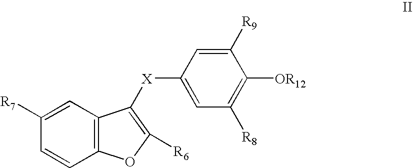
wherein;
R⁶ is C₁-C₄ alkyl;
R⁷ is NHCOR¹⁰, NHCONHR¹⁰, or hydrogen;
R⁸ and R⁹ are independently selected from iodine, and bromine;
R¹⁰ is selected from CF₃, C₁-C₃ alkyl, and (4-R¹¹)C₆H₄;
R¹¹ is selected from C₁-C₄ alkoxy, hydroxy, fluoro, and nitro;
R¹² is selected from hydrogen, and CH₂—COOH;
X is selected from CH₂ and C═O;
   or pharmaceutically acceptable salts and esters thereof and isomers thereof.

Preferably, the compound of formula II is selected from:
2-methyl-3-(3,5-diiodo-4-hydroxy-benzoyl)benzofuran (E5);
2-methyl-3-(3,5-diiodo-4-carboxymethoxy-benzyl)benzofuran (E6);
   and pharmaceutically acceptable salts, esters, and isomers thereof.

Another embodiment of the present invention relates to pharmaceutical compositions for the treatment of atrial fibrillation or atrial flutter comprising at least one compound of formula I or II, if appropriate together with a pharmaceutically-acceptable carrier.

Yet another embodiment of the present invention relates to a method of treating atrial fibrillation or atrial flutter comprising providing to a patient in need thereof a O: pharmaceutically effective amount of at least one compound of formula I or I.

The synthesis and detailed description of the compounds of formula II are described in WO 96/0510 and WO 92/20331.

The compounds of formula I and formula II can be used in combination with other agents ti useful for treating atrial fibrillation and atrial flutter. The individual components of such combinations can be administer separately at different times during the course of therapy or a concurrently in divided or single combination forms. The instant invention is therefore to be understood as embracing all such regimes of simultaneous or alternating treatment and the term “administering” is to be interpreted accordingly., It will be understood that the scope of combinations of the compounds of this invention with other agents useful for treating atrial fibrillation and atrial flutter includes in principle any combination with any pharmaceutical composition useful for treating atrial fibrillation and atrial flutter.

The compounds of formulae I and II can be administered in such oral dosage forms as tablets, capsules (each of which includes sustained release or timed release formulations), pills, powder, granules, elixirs, tinctures, suspensions, syrups and emulsions. Likewise, they may also be administered in intravenous (bolus or infusion), intraperitoneal, topical (e.g., skin cream or ocular eyedrop), subcutaneous, intramuscular, or transdermal (e.g., patch) form, all using forms well known to those of ordinary skill-in the pharmaceutical arts.

The dosage regimen utilizing these compounds is selected in accordance with a variety of factors including type, species, age, weight, sex, and medical condition of the patient; the severity of the condition to be treated; the route of administration; the renal and hepatic function of the patient; and the particular compound or salt thereof employed. An ordinarily skilled physician, veterinarian or clinician can readily determine and prescribe the effective amount of the drug required to prevent, counter or arrest the progress of the condition.

Oral dosages of the compounds, when used for the indicated effects, will range between about 0.01 mg per kg of body weight per day (mg/kg/day) to about 100 mg/kg/day, preferably 0.01 mg per kg of body weight per day (mg/kg/day) to 10 mg/kg/day, and most preferably 0.1 to 5.0 mg/kg/day. For oral administration, the compositions are preferably provided in the form of tablets containing 0.01, 0.05, 0.1, 0.5, 1.0, 2.5, 5.0, 10.0, 15.0, 25.0, 50.0, 100, and 500 milligrams of the active ingredient for the symptomatic adjustment of the dosage to the patient to be treated. A medicament typically contains from about 0.01 mg to about 500 mg of the active ingredient, preferably from about 1 mg to about 100 mg of active ingredient. Intravenously, the most preferred doses will range from about 0.1 to about 10 mg/kg/minute during a constant rate infusion. Advantageously, compounds of the present invention may be administered in a single daily dose, or te total daily dosage may be administered in divided doses of two, three or four times daily. Furthermore, preferred compounds for the present invention can be administered in intranasal form via topical use of suitable intranasal vehicles, or via transdermal routes, using those forms of transdermal skin patches will known to those of ordinary skill in the art. To be administered in the form of a transdermal delivery system, the dosage administration will, of course, be continuous rather than intermittent throughout the dosage regimen.

The specific compounds of formulae I and II described herein can form the active ingredient, and are typically administered in admixture with suitable pharmaceutical diluents, exipients or carriers (collectively referred to herein as “carrier” materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water, and the like. Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms includes sodium oleate, sodium stearate, magnesium stearate, sodium benzoate, sodium acetate, sodium chloride and the like. Disintegrators include without limitation starch, methylcellulose, agar, bentonite, xanthan like.

The compounds of formulae I and II can also be administered in the form of liposome delivery systems, such as small unilamellar vesicles, large unilamellar vesicles and multilamellar vesicles. Liposomes can be formed from a variety of phospholipids, such as 1,2-dipalmitoylphosphatidylcholine, phosphatidyl ethanolamine (cephalin), or phosphatidylcholine (lecithin).

The following definitions apply to the terms as used throughout this specification, unless otherwise limited in specific instances.

The term “alkyl” as employed herein refers to those groups of the designated number of carbon atoms in either a straight and branched chain hydrocarbons, such as methyl, ethyl, propyl, iso-propyl, butyl, isobutyl, tert-butyl, pentyl, hexyl, 2-methylpentyl, and the like.

The term “alkoxy” as employed herein refers to a straight or branched chain radical attached through an oxygen linkage, containing 1, 2, 3 or 4 carbon atoms in the normal chain. Examples of such alkoxy groups are methoxy, ethoxy, propoxy, butoxy, isobutoxy and the like.

The compounds of formulae I and II can be present as salts, in particular pharmaceutically acceptable salts. If they have, for example, at least one basic center, they can form acid addition salts. These are formed, for example, with strong inorganic acids, such as mineral acids, for example sulfueric acid, phosphoric acid or a hydrohalic acid, with strong organic carboxylic acids, such as alkanecarboxylic acids of 1 to 4 carbon atoms which are unsubstituted or substituted, for example, by halogen, for example acetic acid, such as saturated or unsaturated dicarboxylic acids, for example oxalic, malonic, succinic, maleic, fumaric, phthalic or terephthalic acid, such; as hydroxycarboxylic acids, for example ascorbic, glycolic, lactic, malice, tartaric or citric acid, such as amino acids, for example aspartic or glutamic acid or lysine or arginine), or benzoic acid, or with organic sulfonic acids, such as (C₄-C₄)-alkyl- or aryl-sulfonic acids which are unsubstituted or substituted for example by halogen, for example methane- or p-toluene-sulfonic acid. Corresponding acid addition salts can also be formed having, if desired, an additionally present basic center. The compounds of formulae I and II having at least one acid group (for example COOH) can also form salts with bases. Suitable salts with bases are, for example, metal salts, such as alkali metal or alkaline earth metal salts, for example sodium, potassium or magnesium salts, or salts with ammonia or an organic amine, such as morpholine, thiomorpholine, piperidine, pyrrolidine, a mono-, di- or tri-lower alkylamine, for example ethyl-, tert-butyl-, diethyl-, diisopropyl-, triethyl-, tributyl- or dimethyl-propylamine, or a mono-, di- or trihydroxy lower alkylamine, for example mono-, di- or triethanolamine. Corresponding internal salts may furthermore be formed. Salts which are unstable for pharmaceutical uses but which can be employed, for example, for the isolation or purification of free compounds or their pharmaceutically acceptable salts are also included.

Preferred salts of the compounds of formulae I and II which include a basic group include monohydrochloride, hydrogensulfate, tartrate, fumarate or maleate. Preferred salts of the compounds which include an acid group include sodium, potassium and magnesium salts and pharmaceutically acceptable organic amines.

The compounds of formulae I and II may contain one or more chiral centers and therefore may exist as optical isomers. The invention therefore comprises the optically inactive racemic (rac) mixtures (a one to one mixture of enantiomers), optically enriched scalemic mixtures as well as the optically pure individual enantiomers. The compounds in the invention also may contain more than one chiral center and therefore may exist as diastereomers. The invention therefore comprises individual diastereomers as well as mixtures of diastereomers in cases where the compound contains more than one stereo center. The compounds in the invention also may contain acyclic alkenes or oximes and therefore exist as either the E (entgegen) or Z (zusammen) isomers. The invention therefore comprises individual E or Z isomers as well as mixtures of E and Z isomers in cases where the compound contains an, acylic alkene or oxime functional group. Also included within the scope of the invention are polymorphs, hydrates; and solvates of the compounds of the instant invention.

The present invention includes within its scope prodrugs of the compounds of formulae I and II. In general, such prodrugs will be functional derivatives of the compounds of this invention which are readily convertible in vivo into, the required compound. Thus, in the methods of treatment of the present invention, the term “administering” shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example in “Design of Prodrugs” ed. H. Bundgaard, Elsevier, 1985, which is incorporated by reference herein in its entirety. Metabolites of the compounds includes active species produced upon introduction of compounds of this invention into the biological milieu.

The novel compounds of formula I can be prepared according to the following schemes and non-limiting examples, using appropriate materials and are further exemplified by the following non-limiting specific examples. The examples further illustrate details of the preparation of compounds of formula I. Those skilled in the art will readily understand that known variation of the conditions and processes of the following preparative procedures can be used to prepare these compounds.

The compounds of formula I are prepared according to the general methods outlined in Schemes 1 and 2, and according to the methods described. Examples of reagents and procedures for these reactions appear hereinafter and in the working examples.

Compounds of formula I of the invention where X is a carbonyl group (C═O), R₂ is hydrogen, and where variations can be introduced at the R₁, R₃, R₄ and R₅ positions can be prepared using the method outlined below and indicated in Scheme 1 Examples 1 and 2). In the method, benzofuran 1 is regioselective acylated at the β-position by an acyl chloride 2 in the prescence of a Lewis catalyst such as tin tetrachloride, to give the coupled material 3 after standard work-up. A huge collection of different methods for the acylation of aromatics is available in the literature (see for example: Jerry Mach in Advanced Organic Chemistry, 4th ed, 1992, John Wiley & Sons, Inc, p 539-542 and references cited therein), several of which could be applied in the present method.

The methyl ether function can be removed by treatment of 3 with 1-2 equivalents of a Lewis acid such as boron tribromide at low temperature and in an inert solvent such as dichloromethane or benzene. The reaction mixture gives after standard work-up and purification, the end product 4. Several alternative methods for demethylation of anisol derivatives are available in the literature, some which might be applied for the conversion of 3 to 4. Examples of such alternative methods include the use of: (i) AlBr₃/ethanethiol, Node Manubu et al, Tetrahedron Lett., 1989; (ii) BF₃/dimethyl sulfide, Bindal R. D., Katzenellenbogen J. A., J. Oig. Chem, 1987, pp 3181; (iii) HBr/acetic acid, Takeshita Hitosh, Bull. Chem. Soc. Jpn., 1986, pp 1125; and the like.

The phenol 4 is finally O-alkylated employing the appropriate halide in the presence of a base such as potassium carbonate and then further treated with a base, to give the end product containing a carboxymethoxy function. Several alternative methods for the O-alkylation of phenols and hydrolysis of carboxylic acid esters have been published in the litterature, several which might be applied for the conversion of 4 to 5.

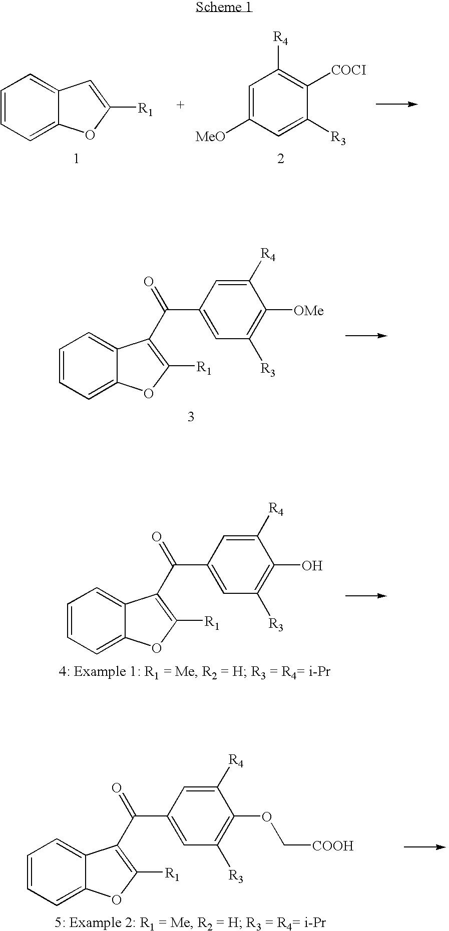

Compounds of formula I of the invention where X is a methylene group (—CH₂—) is hydrogen and where variation can be introduced at the R₁, R₃, R₄, and R₅ positions can be prepared using the method outlined below and indicated in Scheme 2 (Examples 3 and 4). In the method, the carbonyl group (C═O) of 3 is reduced to a methylene group (—CH₂—) employing a combination of lithium aluminum hydride and aluminium trichloride as reducing agent. Several other methods for the reduction of carbonyl groups to methylene groups are known in the litterature and might be used here with successful results and are well known to those skilled in the art (see for example: Jerry March in Advanced Organic Chemistry, 4th ed, 1992, John Wiley & Sons, Inc, p 1209-1211 and references cited therein). The reaction mixture yields after standard work-up the corresponding reduced material 7, which can be further reacted further to give the carboxymethoxy 8 using the same method as described above.

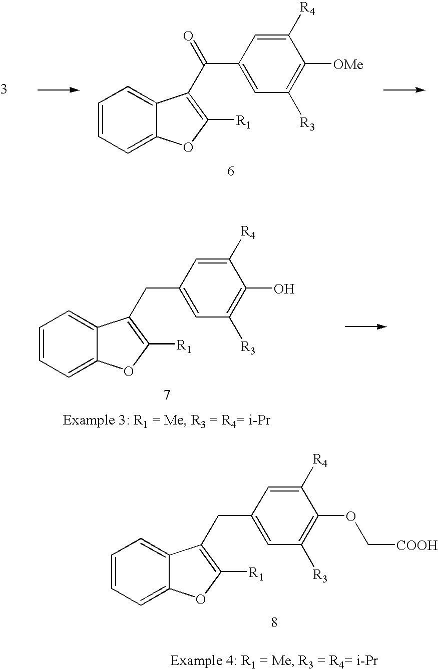

### EXAMPLES

The following Examples represent preferred compounds of formula I of the present invention. However, they should not be construed as lining the invention in any way. The following abbreviations, reagents, expressions or equipment, which are amongst those used in the descriptions below, are explained as follows: gas chromathography mass spectroscopy (GC-MS), electron impact (EI); liquid chromathography mass spectroscopy (LC-MS), electrospray (ES), ethyl acetate (EtOAc).

### Example 1

### 2-methyl-3-(3,5-diisopropyl-4-hydroxybenzoyl)benzofuran (E1)

(a) A stirred mixture of 3,5-diisopropyl-4-methoxybenzoic acid (5 mmol, 1.2 g) and phosporous pentachloride (1.3 g, 6.0 mmol) in dichloromethane (50 mL) was refluxed for two hours. The reaction mixture was cooled down to room temperature, 2-methylbenzofuran (0.76 g, 5 mmol) was added followed by tin tetrachloride (1.3 g, 5 mmol). After two hours the organic solvent was removed and the residue solved in EtOAc, washed with hydrochloric acid (2 N), sodium hydroxide (1 N) and finally with an aqueous saturated solution of sodium chloride. The organic phase was dried over magnesium sulphate. The crude product was purified on column (silica gel, petrolium ether/EtOAc 9:1) to give 1.7 g (97%) of 2-methyl-3-(3,5-diisopropyl-4-methoxybenzoyl)benzofuran as a colorless oil, which slowly solidified at room temperature: ¹H NMR (CD₃COCD₃) d 1.22 (d, 12H, CHCH₃, J=6.9), 2.50 (s, 3H, CH₃), 3.82 (s, 3H, OCH₃), 7.24-7.56 (m, 4H, aromatics), 7.65 (s, 2H, H-2′ and H-6′); MS (ES) m/z 351 (M−1). (b) A stirred solution of 2-methyl-3-(3,5-diisopropyl-4methoxybenzoyl)benzofuran (1.7 g, 4.8 mmol) in 20 mL of dichloromethane was kept under nitrogen and cooled to =40° C. To the solution was added boron tribromide (6.0 mL, 1 N, solution in dichloromethane) and left at room temperature over night. The reaction mixture was treated with cold hydrochloric acid (1 N), the phases were separated and the organic phase was washed once with water. The organic phase was dried over magnesium sulphate, filtrated and concentrated. The residue was subjected to column (silica gel, petrolium ether/EtOAc 8:1) to give 2-methyl-3-(3,5-diisopropyl-4-hydroxybenzoyl)benzofuran as a pale yellow crystal mass (1.3 g, 81%): ¹H NMR (Acetone-d6) d 1.21 (d, 12H, CHCH₃, J=6.9), 2.51(s, 3H, CH₃), 3.41 (m, 1H, CH), 7.57-7.21 (m, 4H, aromatics), 7.64 (s, 2H, H-2′ and H-6′); GC-MS (EI, 70 eV) m/z 336 (M⁺).

### Example 2

### 2-Methyl-3-(3,5-diisopropyl-4-carboxymethoxybenzoyl)benzofuran (E2).

A mixture of 2-methyl-3-(3,5-diisopropyl-4benzofuran (170 mg, 0.5 mmol) and K₂CO₃ (138 mg, 1 mmol) in dry acetone (10 mL), a-brom ethylacetate(170 mg, 1 mmol) was added during 5 minutes, the solution was stirred over night at room temperature. Ethyl acetate was added and the solution was washed with water. The organic phase was evaporated to dryness and the residue was dissolved in a mixture of methanol (2 mL) and sodium hydroxide (2 mL, 1 N). The solution was stirred at room temperature over night, extracted with ethyl acetate and dried over magnesium sulphate. Evaporation of the organic phase gave 1.1 g which was purified on column (silica gel, chloroform/methanol/acetic acid 95:5:1): ¹H NMR (CD₃COCD₃) d 1.21 (d, 12H, CHCH₃, J=6.9), 2.50 (s, 3H, CH₃), 3.49 (m, 1H, CH), 4.56 (s, 2H, CH₂), 7.21-7.61 (m, 4H, aromatics), 7.66 (s, 2H, H-2′ and H-6′); LC-MS (ES) m/z 393(M⁺⁻1).

### Example 3

### 2-Methyl-3-(3,5-diisopropyl-4-hydroxybenzyl)benzofuran (E3)

Aluminium trichloride (120 mg, 4 mmol) in diethyl ether (1.5 mL) was added to a suspension of lithiumaluminiumhydride (40 mg, 2 mmol) in diethyl ether (1 mL) during 20 minutes at 0° C. 2-Methyl-3-(3,5-diisopropyl-4-hydroxybenzoyl)benzofuran (330 mg, 1 mmol) in 3 mL of ether was added, and the mixture then stirred at room temperature for two hours. Excess of the reagent was destroyed b adding water (1 mL) and sodium hydroxide (0.1 mL). Ethyl acetate (100 mL) was added, and the organic layer was washed with sodium bicarbonate and dried over magnesium sulphate. The organic phase was evaporated and the residue and purified on column (petrolium ether/EtOAc 9:1) to give 290 mg (90%) of 2-methyl-3-(3,5-diisopropyl-4-hydroxybenzyl)benzofuran as a red oil: GC-MS (EI, 70 eV) m/z (%) 322(M⁺).

### Example 4

### 2-Methyl-3-(3,5diisopropyl-4-carboxymethoxybenzyl)benzofuran (E4)

This compound was prepared from 2-methyl-3-(3,5-diisopropyl-4-hydroxybenzyl)benzofuran (290 mg, 1 mmol)and a-brom ethylacetate (230 m, 40.5 mmol), using the procedure described in Example 2. The crude product was purified column (chloroform/methanol/acetic acid 95:5:1) to give 300 mg (79%) of 2-methyl-3-(3,5-diisopropyl-4-carboxymethoxy-benzyl)benzofuran as a white crystal mass: ¹H NMR (CD₃COCD₃) d 1.15 (d, 12H, CHCH₃, J=6.9), 2.46 (s, 3H, CH₃), 3.34(m, 1H, CH), 3.97 (s, 2H, CH₂), 4.37(s 2H, CH₂), 7.05-7.45 (m, 4H, aromatics), 7.10(s, 2H,H-2′ and H-6′); LC-MS (ES) m/z 379(M⁺-−1).

The following Table 1 illustrates the potency (IC50-values) of compounds of formulae I and II compared with other anti-arrhythmic drugs to inhibit the transmembrane currents IK(Ado) and IK(ACh) after stimulation of the currents with Adenosine or Acetylcholine (or Carbachol).

TABLE 1
Potency (IC50-values) of compounds of the invention and other
anti-arrhythmic drugs to inhibit the transmembrane currents
IK (Ado) and IK (ACh) after stimulation of the currents with
Adenosine or Acetylcholine (or Carbachol).
IC50: Molar concentration of a compound at which 50%
inhibition of the induced activity occurs.

| | | Inhibition of IK (ACh) |
|---|---|---|
| | Inhibition of IK (Ado) | (Induced by ACh or |
| Compound | Induced by Adenosine | Carbachol) |
| d,l-sotalol (Mori) | No effect | 36 μM (IC50) |
| Propranolol (Brandts) | 8 μM (IC50) | 56 μM (IC50) |
| E-4031 (Mori) | Some effect at 100 μM | 8 μM (IC50) |
| MS-551 (Mori) | Some effect at 100 μM | 11 μM (IC50) |
| Aprinidine (Ohmoto) | Not studied | 0.4 μM (IC50) |
| Amiodarone (Watanabe) | 2 μM (IC50) | 2 μM (IC50) |
| Terikalant (Brandts) | 2 μM (IC50) | 2 μM (IC50) |
| SUN 1165 (Inomata) | Not studied | 29 μM (IC50) |
| Flecainide (Inomata) | Not studied | 3.6 μM (IC50) |
| Disopyramide (Inomata) | Not studied | 1.7 μM (IC50) |
| Quinidine (Inomata) | Not studied | 1.6 μM (IC50) |
| Dronedarone | Not studied | 0.01 μM (IC50) |
| (Guillemare) | | |
| E5 | 1 μM (IC50) | 1 μM (IC50) |
| E6 | Similar to E5 | Similar to E5 |
| | (100% Inh at 50 μM) | (100% Inh at 50 μM) |
| E4 | 100% Inh at 50 μM | 100% Inh at 50 μM |

IC50: Molar concentration of a compound at which 50% inhibition of the induced activity occurs.
E5 is 2-methyl-3-(3,5-diiodo-4-hydroxy-benzoyl)benzofuran. (Formula II)
E6 is 2-methyl-3-(3,5-diiodo-4-carboxymethoxy-benzyl)benzofuran. (Formula II)
E4 is 2-methyl-3-(3,5-diisopropyl-4-carboxymethoxybenzyl)benzofuran. (Formula I)
For references, see Table 2.

TABLE 2
Comparison of blocking activity of E4 and E6 and other antiarrhythmic drugs on
different transmembrane ion-currents.

| | IK(Ado) | IK(ACh) | IK(ATP) | IK1 | IKs | Ito | INa |
|---|---|---|---|---|---|---|---|
| E4, E6 | Yes | Yes | Yes | No | No | No | No |
| Quinidine | U | Yes | Yes | No | No | Yes | Yes |
| | | (Inomata) | (Undrovi) | (Slawsky) | (Lai) | (Slawsky) | (Slawsky) |
| Flecainide | U | Yes | Yes | No | No | Yes | Yes |
| | | (Inomata) | (Sato) | (Slawsky) | (Wang) | (Slawsky) | (Konzen) |
| Disopyramide | U | Yes | Yes | No | Yes | Yes | Yes |
| | | (Inomata) | (Wu) | (Sato) | (Sato) | (Sato) | (Sato) |
| Aprinidine | U | Yes | U | No | No | Yes | Yes |
| | | (Ohmoto) | | (Ohmoto) | (Ohmoto) | (Tanaka) | (Ohmoto) |
| Terikalant | Yes | Yes | No | Yes | Yes | Yes | No |
| (RP58866) | (Brandts) | (Brandts) | (Brandts) | (Yang) | (Yang) | (Yang) | (Yang) |
| d,l-Sotalol | No | Yes (Mori) | U | Yes | No | Yes | No |
| | (Mori) | | | (Berger) | (Lai) | (Berger) | (Malecot) |
| Amiodarone | Yes | Yes | Yes | Yes | Yes | U | Yes |
| | (Watanabe) | (Watanabe) | (Holmes) | (Kodama) | (Kodama) | | (Kodama) |
| Dronedarone | U | Yes | U | U | Yes | U | Yes |
| | | (Guillemare) | | | (Guillemare) | | (Guillemare) |

Explanations

| | |
|---|---|
| Yes: | The compound has been demonstrated to inhibit the particular current |
| | (reference within parenthesis). |
| No: | The compound has been demonstrated to not inhibit the particular current |
| | (reference within parenthesis). |
| U: | No data regarding interaction of the compound with the particular current has |
| | been found in the literature. |
| IK(Ado): | Adenosine activated K-current |
| IK(ACh): | AcetylCholine activated K-current |
| IK(ATP): | ATP-sensitive K-current |
| IK1: | Inward rectifier K-current |
| IKs: | Slow component of the delayed rectifier K-current |
| Ito: | Transient outward K-current |
| INa: | Depolarizing Na-current |

Table 2 References:
Inomata N. et al. Br J Pharmacol 1991 December;104(4):1007-11.
Guillemare E. et al. Marion A, Nisato D, Gautier P. J Cardiovasc Pharmacol 2000 December;36(6):802-5.
Undrovinas A I. et al. Am J Physiol 1990 November;259(5 Pt2):H1609-12.
Slawsky M. T,. And Castle N A. J Pharmacol Exp Ther 1994 April;269(1):66-74.
Lai L. et al. J Biomned Sci 1999 July-August;6(4):251-9.
Satoh H Eur J Pharmacol 2000 Oct. 27;407(1-2):123-9.
Wang D W, et al. Cardiovasc Res 1995 April;29(4):520-5.
Konzen G. et al. Arch Phmacol 1990 J 341(6:56576
Wu B. et al. Cardiovasc Res 1992 November;26(11);1095-101.
Tanaka; H. et al. Naunyn Schmiedebergs Arch Pharmacol 1990 April;341(4):347-56.
Yang B F. et al. Zhongguo Yao Li Xue Bao 1999 November;20(11): 961-9.
Berger F. et al. Arch Pharmacol 1989 December;340(6):696-704.
Holmes D S. Et al. J Cardiovasc Electrophysiol 2000October;11(10):1152-8
Mori K. et al. Circulation 1995 Jun 1;91(11):2834-43.
Ohmoto-Sekine Y. et al. Br J Pharmacol 1999 February;126(3):751-61.
Watanabe Y. et al. J Pharmnacol Exp Ther 1996 November;279(2):617-24.
Brandts B. et al. Pacing Clin Electrophysiol 2000 November;23(11 Pt 2):1812-5.

1. A compound according to formula I; 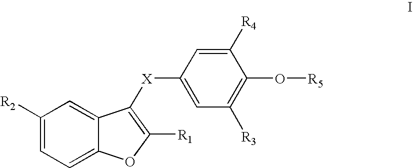
wherein:
R₁ is C₁-C₄ alkyl;
R₂ is NHCOR^{a}, NHCONHR^{a}, or hydrogen;
R₃ and R₄ are independently selected from fluorine, chlorine, C₁-C₆ alkyl, and CF₃;
R^{a} is selected from CF₃, C₁₋₃ alkyl, and -(4-R^{b})C₆H₄;
R^{b} is selected from C₁₋₄ alkoxy, hydroxy, fluoro, and nitro;
R₅ is selected from hydrogen and —CH₂—COOH;
X is selected from CH₂ and C═O; with the proviso that when R₅ is hydrogen, X is —CH₂—;
and pharmaceutically acceptable salts, esters and isomers thereof.

2. A compound according to claim 1 wherein R₂ is hydrogen or NHCOR^{a} and each of R₃ and R₄ is independently C₁-C₄ alkyl.

3. A compound according to claim 2 wherein R₃ and R₄ are isopropyl.

4. A compound according to claim 1 where R₂ is H or NHCOR^{a}, and R₅ is —CH₂—COOH.

5. A compound according to claim 1 wherein R₁ is methyl; R₂ is hydrogen; R₃ and R₄ is C₁-C₄ alkyl; R₅ is —CH₂—COOH; and X is —CH₂—.

6. 2-methyl-3-(3,5-diisopropyl-4-hydroxybenzoyl)benzofuran (E1); or
2-methyl-3-(3,5-diisopropyl-4-carboxymethoxybenzoyl)benzofuran (E2); or
2-methyl-3-(3,5-diisopropyl-4-hydroxybenzyl)benzofuran (E3); or
2-methyl-3-(3,5-diisopropyl-4-carboxymethoxybenzyl)benzofuran (E4); or
and pharmaceutically acceptable salts, esters and isomers thereof.

7. (Cancelled).

8. A pharmaceutical composition comprising a compound according to claim 1, together with a pharmaceutically acceptable carrier.

9. A method of treating atrial fibrillation or atrial flutter comprising providing to a patient in need thereof a pharmaceutically effective amount of a compound according to claim 1.

10-15. (Cancelled).

16. A pharmaceutical composition for the treatment of atrial fibrillation or atrial flutter comprising at least one compound that inhibits certain transmembrane potassium currents, which are more active in the diseased atrium of a mammalian heart than in a normal atrium, without affecting other ion channels.

17. The composition according to claim 16, wherein the said inhibition derives from inhibition of one or several of the three ligand-gated potassium currents IK(Ado), IK(ACh) and IK(ATP).

18. The pharmaceutical composition according to claim 16 wherein the said inhibition caused by the compound is not due to the T3 antagonistic effect.

19. The pharmaceutical composition according to claim 16 wherein the compound is a compound according to formula II 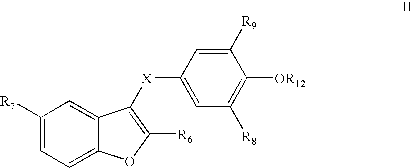
wherein:
R⁶ is C₁-C₄ alkyl;
R⁷ is NHCOR⁵, NHCONHR⁵, or hydrogen;
R⁸ and R⁹ are independently selected from iodine and bromine;
R₁₀ is selected from CF₃, C₁₋₃ alkyl, and 4-R₆C₆H₄;
R¹¹ is selected from C₁₋₄ alkoxy, hydroxy, fluoro, and nitro;
R¹² is selected from hydrogen and —CH₂—COOH;
X is selected from CH₂ and C═O;
or pharmaceutically acceptable salts, esters and isomers thereof.

20. The pharmaceutical composition according to claim 19, wherein the compound is 2-methyl-3-(3,5-diiodo-4-hydroxy-benzoyl)benzofuran (E5); 2-methyl-3-(3,5-diiodo-4-carboxymethoxy-benzyl)benzofuran (E6); or pharmaceutically acceptable salts and esters thereof and isomers thereof.

21. A method of treating atrial fibrillation or atrial flutter comprising providing to a patient in need thereof a pharmaceutically effective amount of at least one compound that inhibits certain transmembrane potassium currents, that are more active in the diseased atrium of a mammalian heart than in a normal atrium, without affecting other ion channels.

22. The method according to claim 21, wherein the said inhibition derives from inhibition of one or several of the three ligand-gated potassium currents IK(Ado), IK(ACh) and IK(ATP).

23. The method according to claim 21 wherein said inhibition caused by the compound is not due to the T3 antagonistic effect.

24. The method according to claim 21 wherein the compound is a compound according to formula II as defined in claim 14. 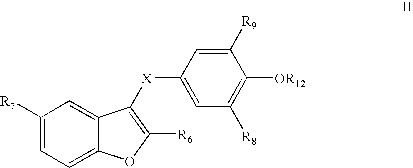
wherein:
R⁶ is C₁-C₄ alkyl;
R⁷ is NHCOR⁵, NHCONHR⁵, or hydrogen;
R⁸ and R⁹ are independently selected from iodine and bromine;
R¹⁰ is selected from CF₃, C₁₋₃ alkyl, and 4-R₆C₆H₄;
R¹¹ is selected from C₁₋₄ alkoxy, hydroxy, fluoro, and nitro;
R¹² is selected from hydrogen and —CH₂—COOH;
X is selected from CH₂ and C═O;
or pharmaceutically acceptable salts, esters and isomers thereof.

25. The method according to claim 21 wherein the compound is
2-methyl-3-(3,5-diiodo-4-hydroxy-benzoyl)benzofuran (ES);
2-methyl-3-(3,5-diiodo-4-carboxymethoxy-benzyl)benzofuran E6);
or pharmaceutically acceptable salts and esters thereof and isomers thereof.
